(19) United States
(12) Patent Application Publication
(10) Pub. No.: US 2013/0184551 A1
(43) Pub. Date: Jul. 18, 2013

(54) NEUROMONITORING DILATOR

(71) Applicants: Jude V. Paganelli, San Diego, CA (US); Corbett W. Stone, San Diego, CA (US); Kabir Gambhir, San Diego, CA (US)

(72) Inventors: Jude V. Paganelli, San Diego, CA (US); Corbett W. Stone, San Diego, CA (US); Kabir Gambhir, San Diego, CA (US)

(21) Appl. No.: 13/350,500

(22) Filed: Jan. 13, 2012

(51) Int. Cl. A61B 5/0492; A61B 5/04

(52) U.S. Cl. 600/377

(57) ABSTRACT

A neuromonitoring dilator including a dilator portion and a probe portion. Each of the dilator portion and the probe portion may be provided with at least one electrode for nerve surveillance. The electrodes may be electrically connectable to a control unit in such a way as to be electrically insulated from each other.

### BACKGROUND OF THE PRESENTLY DISCLOSED INVENTIVE CONCEPTS

1. Field of the Presently Disclosed Inventive Concepts

The inventive concepts disclosed and claimed herein relate to systems and methods for performing surgical procedures and, more particularly, but not by way of limitation, to dilators for accessing a surgical target site to perform surgical procedures.

2. Brief Description of Related Art

The present state of the art, when referencing a lateral surgical access approach, may consist of using the following surgical instruments: neuromonitoring probe, small dilators and larger dilators. After an incision is created, dilators may be used to create a surgical access site which may often be followed by the use of a retractor or other specialized tools creating a surgical access corridor.

During a lateral approach to a patient's spine, a psoas muscle, located on either side of the spine, may be separated in order to access the spine and, in particular, an intervertebral disc space or one or more vertebral bodies within a patient's spinal column. Generally, a surgeon generally tries to avoid nerves of the lumbar plexus that lie within the psoas muscle during such procedures. The anterior third of the psoas muscle is typically considered a safe zone for muscle separation.

To avoid the nerves, surgeons may map the position of the nerves near the psoas muscle using neuromonitoring instruments, such as neuromonitoring probes or neuromonitoring dilators. Generally, neuromonitoring probes and dilators are used in a sequential manner, often in combination with a k-wire. For example, in one process, a neuromonitoring probe may be inserted into the body and a dilator then inserted over the probe. In another known process, a neuromonitoring dilator may first be inserted and then a K-wire inserted through the dilator. With either of these briefly described processes, as well as others, there are numerous steps and instruments involved with obtaining a suitable surgical corridor.

### BRIEF DESCRIPTION OF THE DRAWINGS

FIG. 1 is an elevational view of a neuromonitoring dilator constructed in accordance with the inventive concepts disclosed herein.

FIG. 2 is a sectional view taken along line 2-2 of FIG. 1.

FIG. 3 is an elevational view of the neuromonitoring dilator of FIG. 1 shown inserted within disc space of a spine.

FIG. 4A is a sectional view of another embodiment of a neuromonitoring dilator with a probe portion.

FIG. 4B is a sectional view of the neuromonitoring dilator of FIG. 4A illustrated with another probe portion.

FIG. 5 is an elevational view another embodiment of a neuromonitoring dilator.

FIG. 6 is a sectional view of another embodiment of a neuromonitoring dilator.

### DETAILED DESCRIPTION OF EXEMPLARY EMBODIMENTS

Before explaining at least one embodiment of the presently disclosed and claimed inventive concepts in detail, it is to be understood that the presently disclosed and claimed inventive concepts are not limited in application to the details of construction, experiments, exemplary data, and/or the arrangement of the components set forth in the following description or illustrated in the drawings. The presently disclosed and claimed inventive concepts are capable of other embodiments, or of being practiced or carried out in various ways. Also, it is to be understood that the phraseology and terminology employed herein is for purpose of description and should not be regarded as limiting.

Certain exemplary embodiments of the invention will now be described with reference to the drawings. In general, such embodiments relate to neuromonitoring dilator systems for accessing a patient's spinal column. As generally understood by one of ordinary skill in the art, these systems will be described in connection with accessing the spine to perform a surgical procedure, but the systems will find use not only in orthopedic surgery, but in other surgical procedures in which a surgeon wishes to gain access to an internal cavity by cutting the skin and/or going through a body wall in order to keep the incision spread apart so that surgical instruments may be inserted and associated nerve tissue monitored. For example, the following systems may be used for anteriorly, posteriorly, or laterally accessing the spine, for accessing the thoracic or cervical region of the spine, or for accessing nearly any other part of the body.

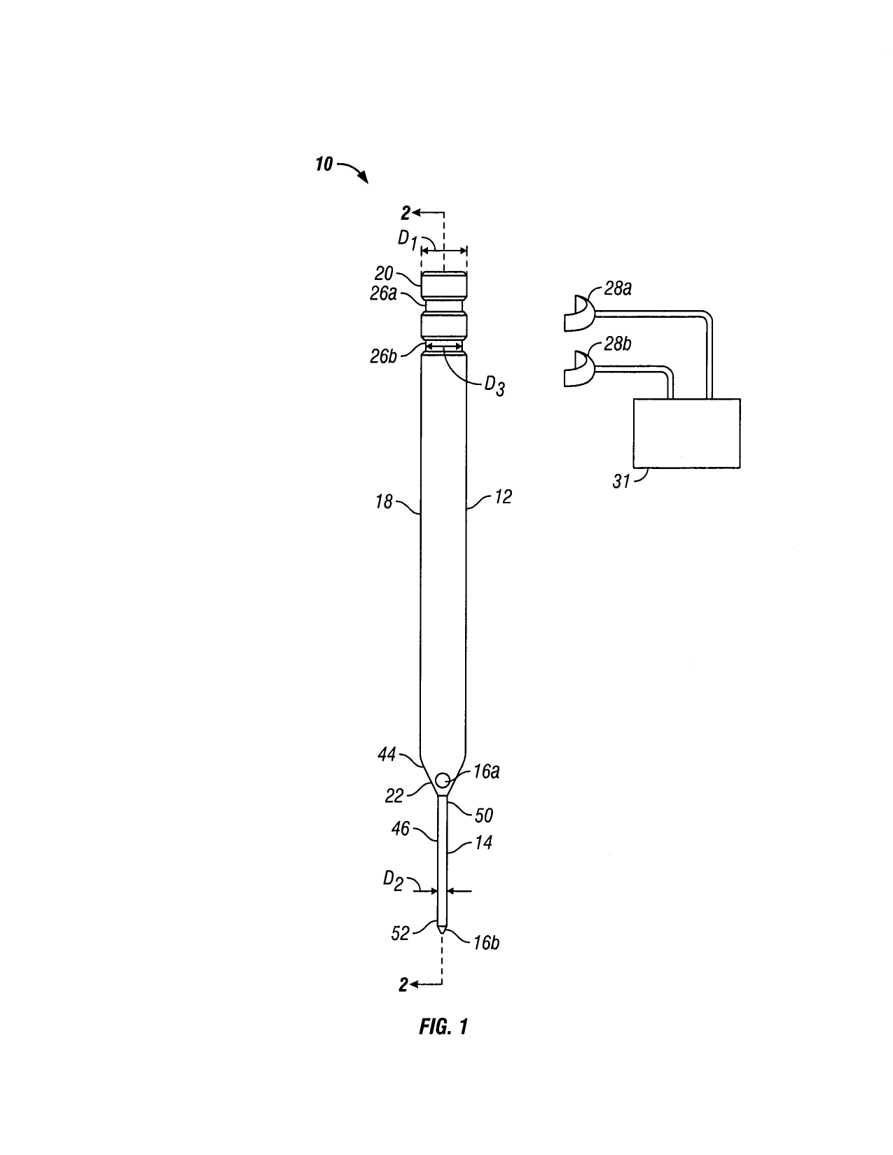

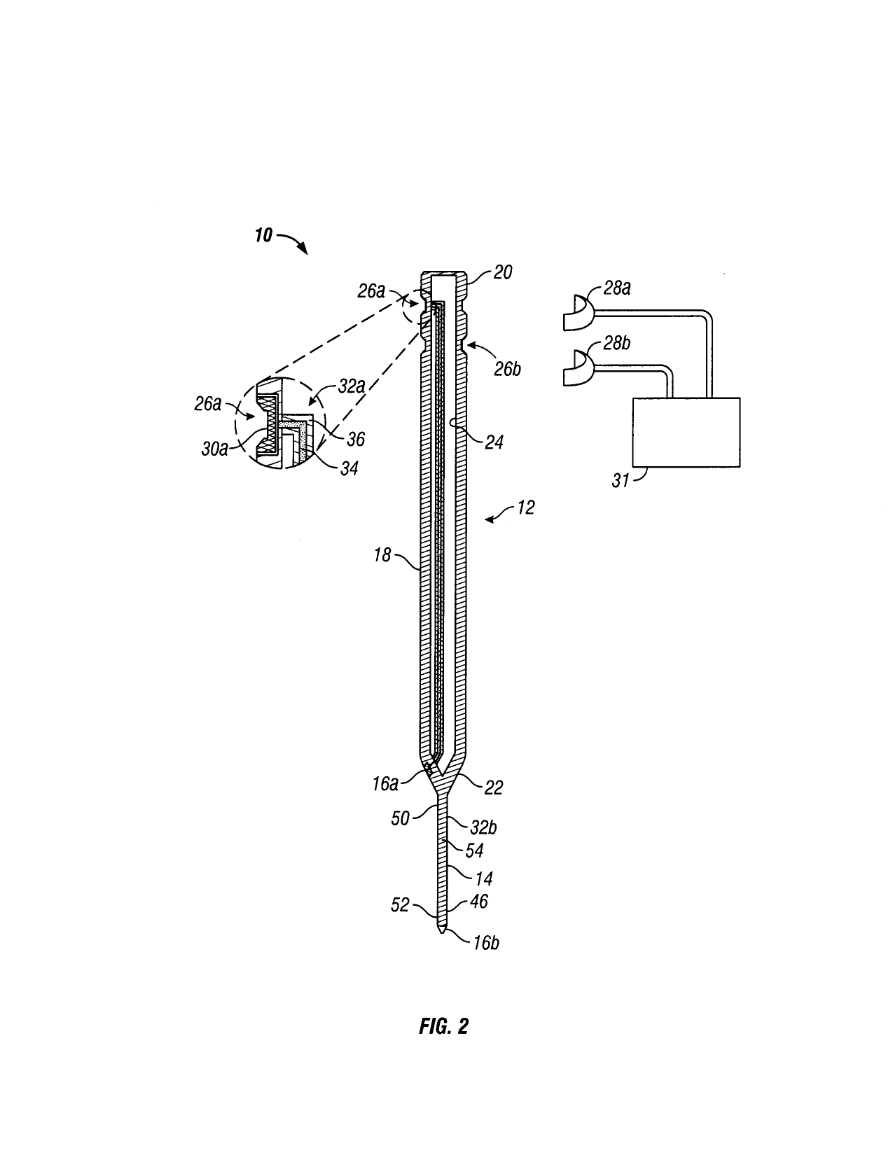

Referring now to the drawings, and more particularly to FIGS. 1 and 2, an embodiment of a neuromonitoring dilator 10 constructed in accordance with the inventive concepts disclosed herein is illustrated. The neuromonitoring dilator 10 may monitor presence and proximity of nerves during introduction into the spine and/or other body part(s), while simultaneously creating and/or enlarging one or more surgical corridors.

In general, the neuromonitoring dilator 10 includes a dilator portion 12 and a probe portion 14 connected to the dilator portion 12 in such a way that the probe portion 14 extends from the dilator portion 12. An axis of the probe portion 14 may be coaxial or substantially coaxial with an axis of the dilator portion 12. Alternatively, the axis of the probe portion 14 may be off-axis from the axis of the dilator portion 12. The dilator portion 12 may be integrally attached to the probe portion 14, or the dilator portion 12 may be removably attached to the probe portion 14, as discussed in more detail herein.

The dilator portion 12 and the probe portion 14 may have different diameters d₁ and d₂, respectively. Generally, the diameter d₁ of the dilator portion 12 may be larger than the diameter d₂ of the probe portion 14. For example, in FIG. 1, the diameter d₁ of the dilator portion 12 is illustrated as significantly larger than the diameter d₂ of the probe portion 14. The diameter d₁ of the dilator portion 12 may be such that, in use, the diameter d₁ of the dilator portion 12 provides expansion of an opening or passage within the body (e.g., surgical corridor). By way of example only, the probe portion 14 may have a diameter in a range of 1-4 mm and the dilator portion 12 may have a diameter in a range of 3-10 mm, and more specifically, the probe portion 14 may have a diameter of about 3 mm and the dilator portion 12 may have a diameter of about 6 mm.

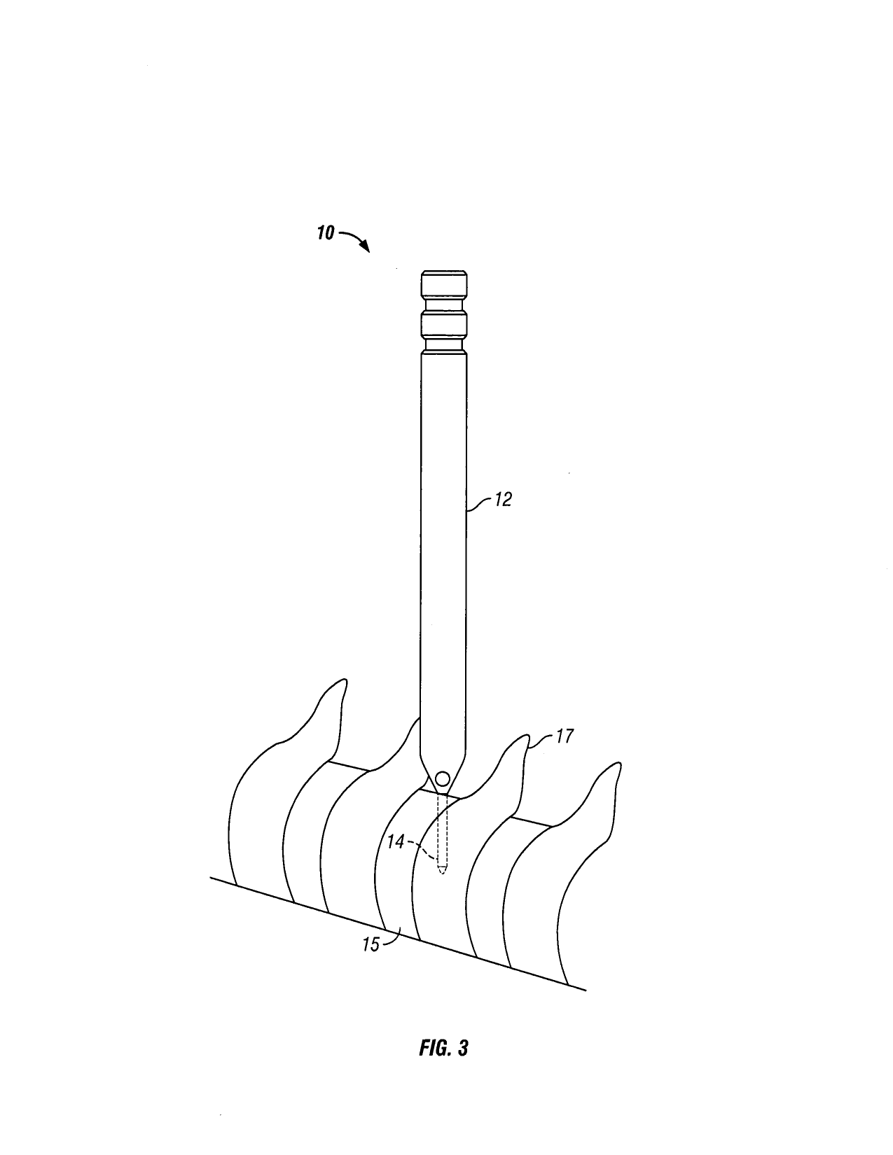

In some embodiments, the diameter d₂ of the probe portion 14 may be generally wider than a standard K-wire used within the industry. Additionally, the diameter d₂ of the probe portion 14 may be such that, in use, the probe portion 14 may be inserted within an intervertebral disc space of the spine. For example, as illustrated in FIG. 3, the diameter d₂ of the probe portion 14 may be between approximately 2-4 mm such that a segment of the probe portion 14 may be inserted into intervertebral disc space 15 of a spine 17.

The dilator portion 12 and the probe portion 14 may each support at least one electrode 16. For example, in FIG. 1, the dilator portion 12 includes an electrode 16a and the probe portion 14 includes an electrode 16b. The electrodes 16a and 16b are provided for the purpose of determining the location of nerves relative to the each of the probe portion 14 and the dilator portion 12 as the neuromonitioring dilator 10 advanced toward the surgical target site. The dilator portion 12 and the probe portion 14 may be equipped with the electrodes via any number of suitable methods, including but not limited to providing electrically conductive elements within the walls of the dilator portion 12 and the probe portion 14 such as by manufacturing the dilators from plastic or similar material capable of injection molding or manufacturing the dilator portion 12 and the probe portion 14 from aluminum (or other suitable metallic substance) and providing outer insulation layer with exposed regions such as by anodizing the exterior of the dilator portion 12 and the probe portion 14.

The electrodes 16 may generally provide neuromonitoring points, assisting in location, proximity, pathology, and direction of nerve tissue. Use of the electrodes 16 and inclusion of both the dilator portion 12 and the probe portion 14 within the neuromonitoring dilator 10 may eliminate one or more steps while accessing the spine or other body parts. For example, use of the electrodes 16 and inclusion of both the dilator portion 12 and the probe portion 14 in the neuromonitoring dilator 10 may cause using an unmonitored k-wire or a monitored EMG probe during the process of accessing the spine or other body parts to be unwarranted.

FIGS. 1 and 2 illustrate one embodiment of the neuromonitoring dilator 10 wherein the dilator portion 12 and the probe portion 14 may be monolithic. To this end, the dilator portion 12 and the probe portion 14 are constructed as one piece from the same material. The dilator portion 12 may include a body 18 having a proximal end 20, a distal end 22, and a lumen 24. The proximal end 20 and the distal end 22 of the dilator portion 12 may be a closed end. In one embodiment, the proximal end 20 of the dilator portion 12 may be closed ended and the distal end 22 of the dilator portion 12 may be an open end.

The proximal end 20 of the body 18 may include one or more clip points 26. The clip points 26 may be conductive contact points for energizing the electrodes 16. For example, the proximal end 20 of the body 18, illustrated in FIGS. 1 and 2, includes two clip points 26a and 26b. The clip point 26a may provide a conductive contact point for energizing the electrode 16a, and the clip point 26b may provide a conductive contact point for energizing the electrode 16b.

The neuromonitoring dilator 10 may include any number of clip points 26 (e.g., 2, 4, 5). For example, the neuromonitoring dilator 10 may include a singular clip point 26 or multiple clip points 26. At least one consideration for determination of the number of the clip points 26 within the neuromonitoring dilator 10 may include use in monitoring presence or proximity of nerves during introduction and advancement toward the spine or other body parts.

The clip points 26 may be positioned on the dilator portion 12 or the probe portion 14. Although the clip points 26 are illustrated on the proximal end 20 of the dilator portion 12 in FIGS. 1 and 2, it should be understood that the clip points 26 may be positioned on any portion of the dilator portion 12. In one embodiment, the clip points 26 may solely energize the electrodes 16 positioned on the dilator portion 12. The electrodes 16 positioned on the probe portion 14 may be energized through other means. For example, the electrodes 16 positioned on the probe portion 14 may be energized through the clip points 26 positioned on the probe portion 14, through a channel external or internal to the dilator portion 12, through a bore in the probe portion 14, and/or the like.

In some embodiments, the clip points 26a and 26b may be reduced diameter portions of the body 18. For example, the diameter d₃ of clip points 26a and 26b may be less than the diameter d₁ of the body 18. Alternatively, the diameter d₃ of one or more of the clip points 26a and 26b may be substantially similar to the diameter d₁ such that the clip points 26 may be flush with the outer surface of the dilator portion 12.

In some embodiments, the clip points 26 may have different diameters d₃ₓ. For example, the diameter d₃, of the clip point 26a may be different than the diameter d₃ₓ of the clip point 26b. Different diameters d₃ₓ for the clip points 26 may provide a visual identification cue for connection of different electrodes 16. For example, a smaller diameter clip point may indicate connection to a first electrode while a larger diameter clip point may indicate connection to a second electrode.

The clip points 26 may include a conductive contact surface 30 sized to receive one or more connectors 28. The connectors 28 may provide a pathway for energy to the electrode 16. For example, the connectors 28 may be connected to a control unit 31 (FIG. 1) in such a way as to deliver energy to the electrodes 16 in an amount sufficient for nerve surveillance. The connectors 28 may include a wiring harness, clip, or other similar mechanism.

The connectors 28 may be capable of being conductively connected to the clip points 26. In some embodiments, the connectors 28 may be conductively connected when forcibly engaged to the contact surface 30 of one or more of the clip points 26. For example, the connector 28a, in FIGS. 1 and 2, may be conductively connected to the clip point 26a when forcibly engaged to the conductive contact surface 30a of the clip point 26a.

Generally, energy may be provided from the clip point 26 to the electrode 16a via a conductive pathway 32 provided within the dilator portion 12. The conductive pathway 32 may be insulated. For example, the clip point 26a may be in conductive communication with the electrode 16a through a first conductive pathway 32a yet insulated from a second conductive pathway 32b which is in the form of the body of the dilator portion 12.

The conductive pathway 32 generally includes insulated conductive elements able to connect with a neuromonitoring station via the connectors 28. The conductive material of the insulated conductive elements may include conductive wiring, conductive epoxy, conductive ink, conductive filaments, and the like. Insulative material of the insulated conductive elements may include any material having low conductivity such that flow of current through is negligible.

The conductive pathway 32 may be positioned within the lumen 24 of the body 18, within the body 18, and/or on the exterior surface of the body 18. For example, as illustrated in FIG. 2, the conductive pathway 32a includes an electrically conductive wire 34 surrounded by an insulator 36 extending from the clip point 26a through the lumen 24 of the body 18 to the electrode 16a.

The electrode 16a can be composed of any suitable biocompatible, electrically conductive material, such as treated aluminum, platinum, platinum/iridium, stainless steel, gold, or combinations or alloys of these materials. In some embodiments, the electrode 16a may utilize the body 18 of the dilator portion as an electrical conductor (i.e., conductive pathway). The dilator portion 12 and the probe portion 12 may be equipped with the electrodes 16 via any number of suitable methods including, but not limited to, providing electrically conductive elements within walls of the body 18 of the dilator portion 12 and the probe portion 14. For example, in FIG. 2, the dilator portion 12 and the probe portion 14 may be formed from plastic (or similar materials capable of injection molding) or from metallic substances, such as aluminum, and providing an outer insulation layer with exposed regions (e.g., anodizing the exterior of the dilator portion 12 and the probe portion 14). In the case of metallic substances, the electrode 16b of the probe portion 14 may utilize the body 18 of the dilator portion 12 and the probe portion 14 as the electrical conductor (i.e., conductive pathway), while the electrode 16a of the dilator portion 12 may utilize a separate conductive pathway (e.g., wire, trace).

In some embodiments, the conductive pathways 32 may be positioned on or within channels of the exterior surface of the body 18 or within the wall of the body 18. For example, a channel may be provided on the exterior surface of the body 18 for housing one or more of the conductive pathways 32. In a similar approach, one or more channels may be provided within the walls of the body 18 or within the lumen 24 for separation and insulation of one or more of the conductive pathways 32.

The conductive pathways 32 provide conductive communication from the clip points 26 to the electrodes 16. For example, the conductive pathway 32a provides conductive communication from the clip point 26a to the electrode 16a. The electrode 16a may be a non-insulated region of the body 18. In some embodiments, the electrode 16 may be a small diameter surface area region on the body 18. For example, the electrode 16a may be a circular area region, as illustrated in FIG. 1. Although the electrode 16a is illustrated as circular, the electrode 16a may be any shape including, but not limited to, circular, oval, triangular, square, or any fanciful shape.

The distal end 22 of the body 18 may include a tapered region 44. The tapered region 44 may house one or more of the electrodes 16. For example, in FIG. 1, the tapered region 44 of the body 18 houses the electrode 16a. In some embodiments, the electrode 16a may circumferentially wrap about the tapered region 44 of the body 18. Although the electrode 16a is illustrated as positioned on the tapered region 44, it should be noted that the electrodes 16 may be positioned on any portion of the body 18.

Additional electrodes 16 may be positioned on the body 18. For example, additional electrodes 16 may be positioned on the proximal end 20 of, or along length of, the body 18. The number and shape of the electrodes 16 may be based on criteria such as aid in determining location, proximity, pathology, and direction of nerve tissue while reducing rotation and movement of the neuromonitoring dilator 10.

Referring to FIGS. 1 and 2, the probe portion 14 may be connected to the distal end 22 of the body 18. The probe portion 14 may include a body 46 having a proximal end 50 and a distal end 52. The proximal end 50 of the probe portion 14 may be connected to the distal end 22 of the body 18. The distal end 52 of the probe portion 14 may have a width such that the probe portion 14 may be insertable into intervertebral disc space, as illustrated in FIG. 3. In some embodiments, the distal end 52 of the probe portion 14 may taper to a point to facilitate insertion.

As described above, the body 46 may be formed of the same material as the body 18 of the dilator portion 12. Alternatively, the body 46 may be formed of a different material depending on the desired rigidity of the probe portion 14 and the desired manner of electrically connecting the electrode 16b.

The body 46 of the probe portion 14 may include one or more of the electrodes 16. For example, in FIGS. 1 and 2, the distal end 52 of the body 46 includes the electrode 16b. As mentioned above, the electrode 16b may be a non-insulated region of the body 46. Alternatively, the electrode 16b can be composed of any suitable electrically conductive material, such as platinum, platinum/iridium, stainless steel, gold, or combinations or alloys of these materials.

The electrode 16b may be any shape including, but not limited to, circular, oval, triangular, square, or any fanciful shape. In some embodiments, the electrode 16b may be a small diameter surface area region located on the distal end 52 of the body 46. In one embodiment, the electrode 16b may be sized and shaped to include substantially all the distal end 52 of the body 46. In one embodiment, the electrode 16b may be positioned on the tip of the distal end 52 of the body 46.

As mentioned above, the connectors 28 are connectable to a control unit 31 (FIG. 1). The control unit 31 can comprise one or more processors capable of executing processor executable code, one or more non-transitory memory capable of storing processor executable code, an input device, and an output device, all of which can be stand-alone, partially or completely network-based or cloud-based, and not necessarily located in a single physical location.

In a one embodiment, the control unit 31 may include a touch screen display. In this embodiment, the touch screen display may form the input device and the output device. The touch screen display may be equipped with a graphical user interface (GUI) capable of communicating information to the user and receiving instructions from the user.

In use, the control unit 31 may be situated outside but close to the surgical field (such as on a cart adjacent to the operating table) such that the touch screen display is directed towards the surgeon for easy visualization. The nuromonitioring dilator 10 accomplishes nerve and neural element sensing by electrically stimulating a retracted nerve root via one or more of the electrodes 16 while monitoring the electromyography (EMG) responses of the muscle group innervated by the particular nerve. The EMG responses provide a quantitative measure of the nerve depolarization caused by the electrical stimulus. Analysis of the EMG responses may then be used to assess the degree to which retraction of a nerve or neural element affects the nerve function over time. One advantage of such monitoring, by way of example only, is that the conduction of the nerve may be monitored during the procedure to determine whether the neurophysiology and/or function of the nerve changes (for better or worse) as the result of the particular surgical procedure. For example, it may be observed that the nerve conduction increases as the result of the operation, indicating that the previously inhibited nerve has been positively affected by the operation.

A method of using the neuromonitoring dilator 10 illustrated in FIGS. 1-3 will now be described for accessing a patient's spine. In some embodiments, technique of use may be used as an alternative to existing pedicle probes for preparing and/or testing a pedicle screw for fixation, although a similar or the same method may be used in other parts of the patient's body. The neuromonitoring dilator 10 may provide access to a surgical site by monitoring presence and proximity of nerves during introduction while creating and/or enlarging a surgical corridor through tissue.

A surgeon may identify an operative level and create an incision for introduction of the neuromonitoring dilator 10 into the patient's body. The connectors 28 are attached to the clip points 26 to establish electrical communication between the neuromonitoring dilator 10 and the control unit 31. The surgeon may advance the neuromonitoring dilator 10 through the patient's body towards a surgical target site. For example, the surgeon may advance the neuromonitoring dilator 10 towards disc space 15 of the spine 17. The psoas muscle located on either side of the spine may be initially engaged by the distal end 52 of the probe portion 14. The distal end 52 of the probe portion 14 may provide an initial separation of the psoas muscle. The dilator portion 12 may further enlarge this separation providing a surgical corridor.

During advancement, the surgeon may use the neuromonitoring dilator 10 to map nerve location. Neural elements and nerves of the psoas muscle may be mapped using the electrodes 16 positioned on the dilator portion 12 and the probe portion 14. For example, the connectors 28 may provide energy to the electrodes 16 positioned on the dilator portion 12 and the probe portion 14 through the conductive pathways 32. The conductive pathways 32 within the dilator portion 12 and the probe portion 14 may then provide electrical stimulation of nerves via one or more of the electrodes 16. Electromyography (EMG) responses of muscle group reactions may be recorded based on electric currents from active muscle stimulated by nerves. These EMG responses may provide a quantitative measure of nerve depolarization for which presence, proximity, pathology, and direction of the nerves may be determined. Presence, proximity, pathology, and direction may provide the surgeon a map of nerve location.

The map may also provide the surgeon a map of a safe zone, i.e., a zone generally free of neural elements or nerves, on a tissue of interest. For example, the most posterior neural or nerve free area of the psoas muscle may be located and identified. The distal end 52 of the probe portion 14 may then be inserted through the psoas muscle via the nerve free tissue area, or through nearly any other region free of neural elements or nerves, and into the intervertebral disc space 15 of the spine 17. The probe portion 14 is further inserted into the disc space 15 until the distal end 44 of dilator portion 12 is positioned proximate the disc space 15. At this point, the connectors 28 may be removed and additional dilators positioned over the dilator portion 12.

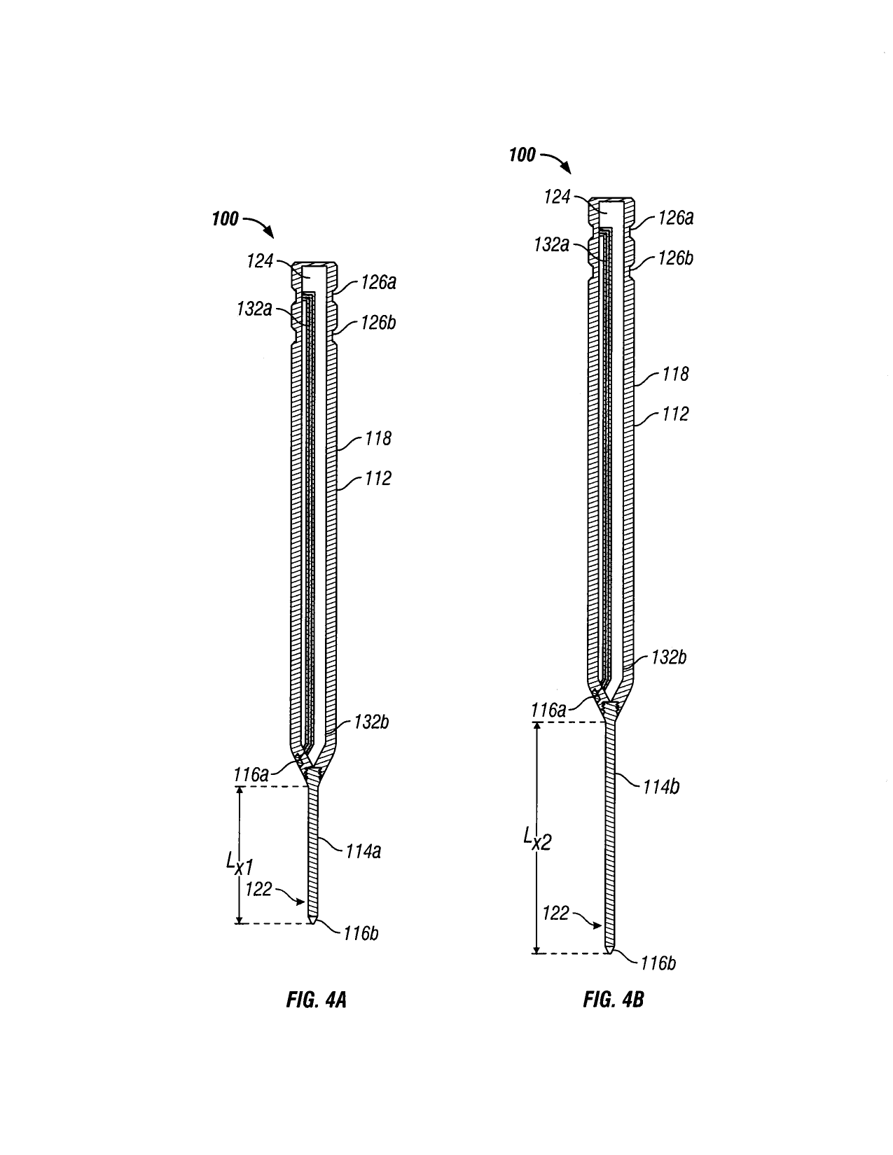

FIGS. 4A and 4B illustrate another embodiment of a neuromonitoring dilation neuromonitoring dilator 100. The neuromonitoring dilator 100 may be similar to the neuromonitoring dilator 10 illustrated in FIGS. 1 and 2; however, a probe portion 114 may be detachably connected to a dilator portion 112. In some embodiments, one or more probe portions 114 may be manufactured as disposable or reusable instruments within the neuromonitoring dilator 100.

The probe portion 114 may be detachably connected to the dilator portion 112 in a variety of manners, such as by screw fit, press fit, snap fit, or combinations thereof. For example, in FIG. 4A and 4B, the probe portions 114a and 114b are illustrated as being threadingly connected to the dilator portion 112. Alternatively, the probe portion 114 may be semi-permanently attached to the dilator portion 112 using adhesive or bonding-type material. With the probe portion 114 detachably connected to the dilator portion 112, different probe portions 114 may be interchangeable with the dilator portion 112.

Each probe portion 114 interchangeable with the dilator portion 112 may have a fixed length Lₓ. The fixed length Lₓ may be any length determined suitable for use. For example, the fixed length Lₓ may be any length determined suitable for accession and/or monitoring presence and/or proximity of nerves during introduction and accession into the spine and/or other body part. As illustrated in FIGS. 4A, a first probe 114a has a fixed length Lₓ₁. In FIG. 4B, the first probe 114a has been replaced by a second probe 114b having a fixed length Lₓ₂ wherein Lₓ₁≠Lₓ₂.

In one embodiment, a kit may include at least one dilator portion 112 and one or more probe portions 114. The probe portions 114 may have similar lengths Lₓ and/or different lengths with each probe portion 114 able to be removably received within the dilator portion 112.

In use, a surgeon may determine a suitable length Lₓ of the probe portion 14 prior to advancement of the neuromonitoring dilator 100 in the patient's body. As such, the probe portion 114 may be fit to the dilator portion 112 prior to advancement of the neuromonitoring dilator 100 in the body. Suitable length Lₓ may be based on a variety of factors including, but not limited to, surgery type, patient anatomy, and level and direction of surgical approach.

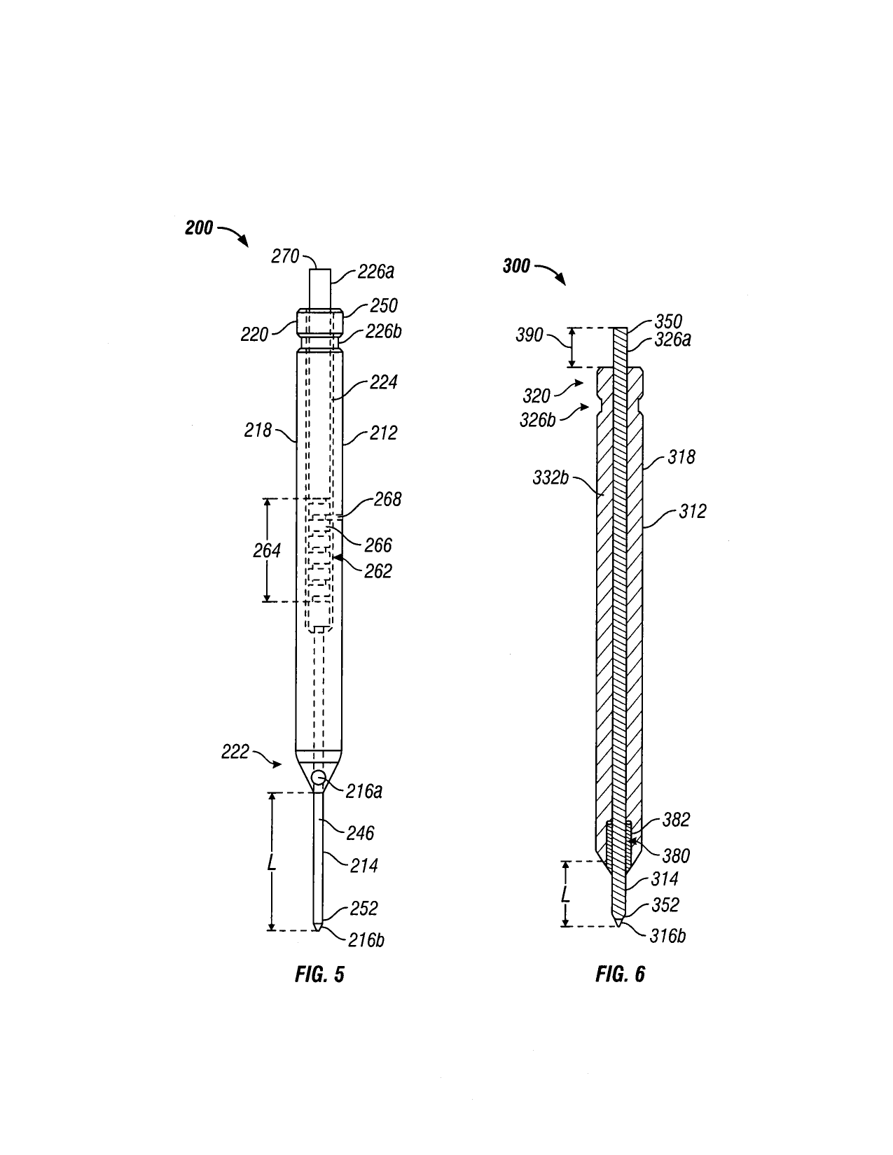

FIG. 5 illustrates another embodiment of a neuromonitoring dilator 200. The neuromonitoring dilator 200 includes an internal mechanism to advance a probe portion 214 from a lumen 224 of a dilator portion 212. The probe portion 214 may be slidably connected to the dilator portion 212 in such a way that the distance which a distal end 252 of the probe portion 214 is positioned from a distal end 222 of the dilator portion 212 may be selectively adjustable.

A body 246 of the probe portion 214 may extend at least the length of the lumen 224 through the dilator portion 212. For example, a proximal end 220 of the dilator portion 212 and a proximal end 250 of the probe portion 214 may terminate at substantially similar positions as illustrated in FIG. 5.

A length L of the probe portion 214 may be positioned outside of the lumen 224 extending outward from the distal end 222 of the dilator portion 212. The length L of probe portion 214 may be increased or decreased using an internal or external mechanism, such as an indexing mechanism or a ratcheting mechanism. For example, FIG. 5 illustrates an indexing mechanism 262 for changing the length L of the probe portion 214. The indexing mechanism 262 may be housed in the dilator portion 212 in such a way as to selectively advance or retract the probe portion 214 relative to the dilator portion 212. For example, the indexing mechanism 262 may be used to propel the probe portion 214 from the lumen 224 of the dilator portion 212, thus increasing the length L of the probe portion 214.

The indexing mechanism 262 may include a substantially linear portion 264 of the probe portion 214. The substantially linear portion 264 of the probe portion 214 may include two or more teeth 266. In some embodiments, the teeth 266 may be substantially similar in design (e.g., square, triangular, or the like). Additionally, the teeth 266 may be spaced adjacent to one another. Spacing may be substantially similar or different depending on adjustment variables for the length L.

The indexing mechanism 262 may also include a detent 268 housed in the dilator portion 212 so as to cooperate with the teeth 266 to hold the probe portion 214 in a selected position. The detent 268 may be any suitable mechanism, such as a spring-loaded ball. In use, the probe portion 214 may be manipulated at a proximal end 270 of the probe portion 214 allowing the probe portion 214 to slidably advance through the lumen 224.

In one embodiment, the probe portion 214 may be energized via the proximal end 270 of the probe portion 214. For example, a portion or the entire proximal end 270 may be formed of conductive material and serve as a clip point 226a for a conductive pathway. Referring to FIGS. 2 and 5, the connector 28a may be attached to the proximal end 270. For example, the connector 28a may be forcibly engaged about the proximal end 270 providing energy to the conductive material of the probe portion 214. A bore may extend the length of the probe portion 214 from the proximal end 250 to the distal end 252 such that a conductive pathway may be positioned within the bore providing energy from the connector 28a to the electrode 216b.

The electrode 216a positioned on the dilator portion 212 may be energized via the clip point 226b. The clip point 226b may be in conductive communication with the electrode 216a through a conductive pathway including, but not limited to, conductive wiring, conductive epoxy, conductive ink, conductive filaments, and the like.

In some embodiments, the probe portion 214 may be advanced through the lumen 224 manually and secured by a locking element (e.g., pin, screw, or other similar mechanism). The probe portion 214 and the dilator portion 212 may have a slip fit with the probe portion 214 slidably engaged within the dilator portion 212. The locking element may be used to secure the probe portion 214 inside the dilator portion 212 using a friction fit. When the locking element is disengaged, the probe portion 214 may be manually adjusted by an operator such that a desired length of the probe portion 214 may be provided. Once the desired length for the probe portion 214 is provided, the locking element may be adjusted and engaged to secure the probe portion 214 inside the dilator portion 212 by friction fit.

FIG. 6 illustrates another embodiment of a neuromonitoring dilator 300. The neuromonitoring dilator 300 includes a collet system 380. The collet system 380 may engage a probe portion 314 within a dilator portion 312.

The collet system 380 may include a clamp 382. In some embodiments, the clamp 382 may be cylindrical. The clamp 382 may, however, be any shape including triangular, square, or any fanciful shape. In some embodiments, the clamp 382 may include a slotted design with corresponding mating parts positioned within a lumen 324 of the dilator portion 312.

In use, the clamp 382 may be slip fit within the lumen 324 through a distal end 322 of the dilator portion 312. The clamp 382 may be capable of gripping the probe portion 314. For example, the clamp 382 may grip the probe portion 314 when the clamp 382 is compressed by a body 318 of the dilator 312.

In some embodiments, the probe portion 314 may extend beyond a proximal end 320 and the distal end 322 of the dilator portion 312. For example, as illustrated in FIG. 6, the dilator portion 312 may include an open sided proximal end 320 and an open sided distal end 322. A segment 390 of the probe portion 314 may extend beyond the proximal end 320 of the dilator portion 312.

The probe portion 314 may be energized via the segment 390 of the probe portion 314 extending beyond the proximal end 320 of the dilator portion 312. For example, the segment 390 of the probe portion 314 extending beyond the proximal end 320 of the dilator portion 312 may include conductive material and serve as a clip point 326a for a conductive pathway. Referring to FIGS. 2 and 6, the connector 28a may be attached to the segment of the probe portion 314. For example, the connector 28a may be forcibly engaged about the segment 390 providing energy to the conductive material within the segment 390. A bore may extend the length of the probe portion 314 from the proximal end 350 to the distal end 352 such that a conductive pathway may be positioned within the bore conductively providing energy from the connector 28a to an electrode 316b.

Alternatively, the probe portion 314 may provide electrically conductive elements within the walls of the probe portion 314 to provide energy from the connector 28a to the electrode 316b. For example, the probe portion 314 may be formed by manufacturing plastic (or similar materials capable of injection molding), or formed by manufacturing aluminium, or other similar metallic elements, and providing an outer insulation layer with exposed regions (e.g., anodizing the exterior of the aluminium probe portion 314). As such, the electrode 316b of the probe portion 314 may utilize the probe portion 314 as the electrical conductor (i.e., conductive pathway).

The electrode 316b may be electrically insulated from an electrode 316a of the dilator portion 312. In some embodiments, the electrode 316a may utilize the body 318 of the dilator portion 312 as an electrical conductor (i.e., conductive pathway). For example, the dilator portion 312 may provide electrically conductive elements within the walls of the body 318 similar to the probe portion 314. As such, the electrode 316a of the dilator portion 312 may utilize the body 18 of the dilator portion 312 as the electrical conductor (i.e., conductive pathway) and be electrically insulated from the electrode 316b.

From the above description, it is clear that the inventive concepts disclosed and claimed herein are well adapted to carry out the objects and to attain the advantages mentioned herein, as well as those inherent in the invention. While exemplary embodiments of the inventive concepts have been described for purposes of this disclosure, it will be understood that numerous changes may be made which will readily suggest themselves to those skilled in the art and which are accomplished within the spirit of the inventive concepts disclosed herein.

What is claimed is:

1. A neuromonitoring dilator, comprising:
a dilator portion having a proximal end, a distal end, and a diameter, the distal end of the dilator portion being tapered and provided with at least one electrode, the electrode being electrically connectable to a control unit in such a way as to deliver energy to the electrode in an amount sufficient for nerve surveillance; and
a probe portion connected to the dilator portion in such a way that the probe portion extends from the distal end of the dilator portion, the probe portion having a distal end and a diameter, the diameter of the probe portion being less than the diameter of the dilator portion, and the distal end of the distal end of the probe portion provided with at least one electrode, the electrode being electrically connectable to the control unit in such a way as electrically insulate the electrode of the probe portion from the electrode of the dilator portion and to deliver energy to the electrode of the probe portion in an amount sufficient for nerve surveillance.

2. The neuromonitoring dilator of claim 1, wherein the probe portion has a width such that the probe portion is insertable into an intervertebral disc space.

3. The neuromonitoring dilator of claim 2, wherein the distal end of the probe portion tapers to a point.

4. The neuromonitoring dilator of claim 1, wherein the dilator portion and the probe portion are monolithic.

5. The neuromonitoring dilator of claim 1 wherein the probe portion is detachably connected to the dilator portion.

6. The neuromonitoring dilator of claim 1 wherein the probe portion is slidably connected the dilator portion in such a way that the distance which the distal end of the probe portion is positioned from the distal end of the dilator portion is selectively adjustable.

7. The neuromonitoring dilator of claim 7, further comprising an indexing mechanism housed in the dilator portion in such a way as to selectively advance the probe portion relative to the dilator portion.

8. A neuromonitoring dilator kit, comprising:
a dilator portion having a proximal end, a distal end, and a diameter, the distal end of the dilator portion being tapered and provided with at least one electrode, the electrode being electrically connectable to a control unit in such a way as to deliver energy to the electrode in an amount sufficient for nerve surveillance; and
a plurality of probe portions, each of the probe portions having a length different from the length of the other probe portion and each probe portion being detachably connectable to the dilator portion in such a way that the probe portions are interchangeably extendable from the distal end of the dilator portion, each of the probe portions having a distal end and a diameter, the diameter of each of the probe portions being less than the diameter of the dilator portion, and the distal end of each of the probe portions provided with at least one electrode, the electrode of each of the probe portions being electrically connectable to the control unit in such a way as to electrically insulate the electrode of the probe portion from the electrode of the dilator portion and to deliver energy to the electrode of the probe portion in an amount sufficient for nerve surveillance when the probe portion is connected to the dilator portion.

9. The kit of claim 9, wherein a proximal end of each of the probe portions is threadingly connectable to the distal end of the dilator portion.

10. The kit of claim 8, wherein each of the probe portions has a width such that the probe portion is insertable into an intervertebral disc space.

11. The kit of claim 11, wherein the distal end of at least a portion of the probe portions tapers to a point.

12. A method of accessing a surgical target site while protecting adjacent nerves, comprising:
providing an incision in a body;
inserting and advancing a neuromonitoring dilator to the surgical target site within the body, the neuromonitoring dilator comprising:
a dilator portion having a proximal end, a distal end, and a diameter, the distal end of the dilator portion being tapered and provided with at least one electrode for nerve surveillance; and
a probe portion connected to the dilator portion in such a way that the probe portion extends from the distal end of the dilator portion, the probe portion having a distal end and a diameter, the diameter of the probe portion being less than the diameter of the dilator portion;
electrically stimulating the electrodes of the dilator portion and the probe portion;
receiving electromyography responses from the body based on the electrical stimulation provided by the electrodes of the neuromonitoring dilator; and
mapping nerve location while simultaneously enlarging a surgical corridor to the target site.

13. The method of claim 12 further comprising adjusting the distance the distal end of the probe portion extends from the distal end of the dilator portion.

14. The method of claim 12 further comprising inserting the probe portion into the surgical target site until the distal end of the dilator portion is positioned proximate the surgical target site.

15. The method of claim 14, wherein the surgical target site is an intervertebral disc space.
